(19) United States
(12) Patent Application Publication
(10) Pub. No.: US 2008/0207716 A1
(43) Pub. Date: Aug. 28, 2008

(54) Formulations and Dosing Regiment for Ppar-Alpha Modulators

(75) Inventors: Eyas Jamal Abu-Raddad, Indianapolis, IN (US); Michael Arthur Derby, Avon, IN (US); Cynthia Joyce Harris, Indianapolis, IN (US); Daniel Charles Howey, Indianapolis, IN (US); Laura Frey Michael, Indianapolis, IN (US)

Correspondence Address: ELI LILLY & COMPANY, PATENT DIVISION, P.O. BOX 6288, INDIANAPOLIS, IN, 46206-6288, US

(73) Assignee: Eli Lilly and Company Patent Division, Indianapolis, IN (US)

(21) Appl. No.: 11/814,487

(22) PCT Filed: Jan. 25, 2006
(86) PCT No.: PCT/US06/02606

§ 371 Date: Jul. 23, 2007

Related U.S. Application Data
(60) Provisional application No. 60/647,903, filed on Jan. 28, 2005.

(51) Int. Cl. A61K 31/4196; A61P 3/00; A61P 25/00; A61P 9/00; A61P 35/00

(52) U.S. Cl. 514/384

(57) ABSTRACT

The present invention provides dosing regimens for PPAR-alpha agonist and compositions thereof. A further embodiment of the present invention is a solid oral formulation of Propanoic Acid, 2-[4-[3-[2,5-dihy-dro-1-[(4-methylphenyl)methyl]-5-oxo-1H-1,2,4-triazol-3-yl]propyl]phenoxyl-2-methyl- comprising 75 μg or less of Propanoic Acid, 2-[4-[3-[2,5-dihydro-1-[(4-methylphenyl)methyl]-5-oxo-1H-1,2,4-triazol-3-yl]propyl]phenoxy]-2-methyl- per dosage unit.

### FIELD OF THE INVENTION

This invention relates to methods for treating diseases or conditions with PPAR-alpha modulators. In particular, this invention relates to dosing regimens for PPAR-alpha agonist and compositions thereof. A further embodiment of the present invention is an oral formulation of Propanoic Acid, 2-[4-[3-[2,5-dihydro-1-[(4-methylphenyl)methyl]- 5-oxo-1H-1,2,4-triazol-3-yl]propyl]phenoxy]-2-methyl-comprising 75 μg or less of Propanoic Acid, 2-[4-[3-[2,5-dihydro-1-[(4-methylphenyl)methyl]-5-oxo-1H-1,2,4-triazol-3-yl]propyl]phenoxy]-2-methyl- per dosage unit.

### BACKGROUND OF THE INVENTION

PPAR-alpha agonists are being studied for use to treat or prevent cardiovascular conditions and/or dyslipidemia. PPAR-alpha agonist compounds are known in the art. For example, WO 02/038,553 (herein “the '553 patent”) discloses PPAR agonist compounds, methods for preparing, formulations, and uses therefore. However, applicants are aware of no publications teaching less than daily oral dosing for selective PPAR alpha agonist compounds.

The '553 publication teaches that the PPAR modulators described therein can be administered using a daily dosing regimen. The regimen may include a total daily dose administered in divided doses during the day; however, the dosing regimen does not direct the artisan to utilize intentional less than daily periodic dosing.

Drug dosing regimens are typically designed to be convenient for the patient, to promote compliance, to produce maximal benefit, and to minimize undesired side effects. For most drugs, this is best achieved through constant drug effect at the lowest level. Daily drug administration is often required to achieve a constant drug effect. For drugs given daily, efficacy and side effects are generally separable by total drug dosage, with side effects occurring at drug dosages that are higher by some multiple than the dosage producing the desired effect.

Many patients prefer to take medications only once-weekly, once-monthly, or every other week for increased convenience, improved dosing compliance, and to minimize patient drug exposure. The present invention fulfills the patient's desire for a convenient less-than daily dosing regimen for PPAR alpha agonist. The dosing regimen provided by the present invention may offer patients a more pharmaceutically desirable safety profile. Further, the surprisingly high potency of the LY518674 compound may not enable administration of a lower dosage of the compound for a patient desiring a lower dose. The less frequent dosing regimen provided herein offers such patients a desirable dosing option that allows the patient to customize or titrate their dose.

In addition to enhanced patient safety, minimization of drug and health provider cost is sometimes part of the rationale for less than daily dosing. Additionally, less than daily oral drug administration can be desirable for patients having difficulty swallowing pills. The dosing regimen provided herein offers patients a dosing option with minimal drug exposure, convenience, and the potential for enhanced health economics outcomes.

The present invention further provides a clinically desirable formulation comprising about 75 μg or less of Propanoic Acid, 2-[4-[3-[2,5-dihydro-1-[(4-methylphenyl)methyl]-5-oxo-1H-1,2,4-triazol-3-yl]propyl]phenoxy]-2-methyl or a pharmaceutically acceptable salt thereof. The formulation offers a pharmaceutically acceptable, consistent dose, and effective drug delivery of the Propanoic Acid, 2-[4-[3-[2,5-dihydro-1-[(4-methylphenyl)methyl]-5-oxo-1H-1,2,4-triazol-3-yl]propyl]phenoxy]-2-methyl pharmaceutically active agent.

### DESCRIPTION of the DRAWINGS

FIG. 1. FIG. 1 illustrates the time profile of percentage change from baseline for LDL-C following the administration of daily oral doses of LY518674 over 14 days and 30 day follow up afterwards, in healthy obese subjects in MDSS of a study conducted substantially as described by Example 1.

FIG. 2. FIG. 2 illustrates simulated time profiles of percentage change from baseline for LDL-C following the administration of LY518674 according to different dosing regimens and placebo as described by Example 4. The total dose administration is similar across regimens. The model used to generate the simulations was based on the results of an MDSS clinical study conducted substantially as described in Example 1.

### SUMMARY OF THE INVENTION

The present invention provides a once monthly dosing regimen for PPAR alpha modulators.

The present invention provides a once monthly dosing regimen for

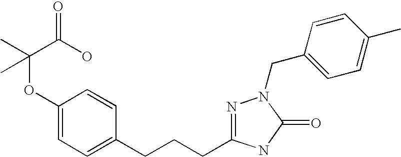

or a pharmaceutical salt thereof.

In a further embodiment of the present invention, a twice-monthly dosing regimen for PPAR alpha modulators is provided. The present invention provides a twice-monthly dosing regimen for

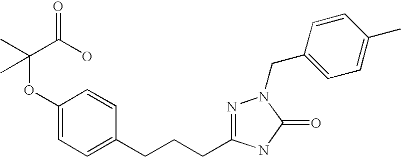

or a pharmaceutical salt thereof.

Further, the present invention provides a pharmaceutically effective bi-weekly dosing regimen for PPAR alpha compounds. The present invention provides a bi-weekly dosing regimen for

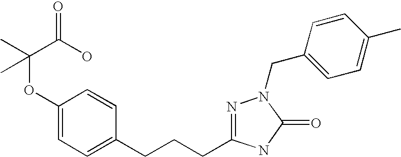

or a pharmaceutical salt thereof.

Briefly, in one aspect, the present invention provides a method of treating a disease or condition, for example, hyperlipidemia, with a PPAR-alpha agonist comprising administration of the PPAR-alpha agonist according to a dosing schedule that comprises at least one period of less than daily dosing of the agonist, for example alternate day dosing or once every other week or one time per month. The most preferred PPAR-alpha agonist is LY518674.

A further embodiment of the present invention is a formulation for daily or less than daily dosing wherein the active PPAR alpha agonist is formulated to contain about 75 μg or less per dosage unit. In a further embodiment it may be preferred that the dosage unit contains about 50 μg or less per dosage unit. A further desired embodiment is a formulation containing about 25 μg or less per dosage unit. The active PPAR alpha agonist that is preferred for the oral dosage form is Propanoic Acid, 2-[4-[3-[2,5-dihydro-1-[(4-methylphenyl)methyl]-5-oxo-1H-1,2,4-triazol-3-yl]propyl]phenoxy]-2-methyl-. Orally administered Propanoic Acid, 2-[4-[3-[2,5-dihydro-1-[(4-methylphenyl)methyl]-5-oxo-1H-1,2,4-triazol-3-yl]propyl]phenoxy]-2-methyl- has now demonstrated surprising potency in human patients. It may be especially preferred that the oral dosage form is provided as a solid. It can be especially desirable to provide the patient with the smallest effective dose to provide maximal therapeutic benefit while minimizing any undesired side effects that may appear in a given patient taking the drug.

Another feature of the present invention is a solid formulation comprising about 25 μg or less Propanoic Acid, 2-[4-[3-[2,5-dihydro-1-[(4-methylphenyl)methyl]-5-oxo-1H-1,2,4-triazol-3-yl]propyl]phenoxy]-2-methyl- per dosage unit and one or more pharmaceutically acceptable carriers or excipients. Another feature of the present invention is a solid oral formulation comprising about 25 μg or less Propanoic Acid, 2-[4-[3-[2,5-dihydro-1-[(4-methylphenyl)methyl]-5-oxo-1H-1,2,4-triazol-3-yl]propyl]phenoxy]-2-methyl- per dosage unit and one or more pharmaceutically acceptable carriers or excipients. A further feature of the present invention is an oral formulation comprising 25 μg or less Propanoic Acid, 2-[4-[3-[2,5-dihydro-1-[(4-methylphenyl)methyl]-5-oxo-1H-1,2,4-triazol-3-yl]propyl]phenoxy]-2-methyl- per dosage unit and one or more pharmaceutically acceptable carriers or excipients. It may be especially desired that each dosage unit contains from about 5 μg to about 25 μg of Propanoic Acid, 2-[4-[3-[2,5-dihydro-1-[(4-methylphenyl)methyl]-5-oxo-1H-1,2,4-triazol-3-yl]propyl]phenoxy]-2-methyl- . It may also be preferred that the dosage unit contains from about 10 μg to about 25 μg Propanoic Acid, 2-[4-[3-[2,5-dihydro-1-[(4-methylphenyl)methyl]-5-oxo-1H-1,2,4-triazol-3-yl]propyl]phenoxy]-2-methyl-. The oral formulation will generally be provided as a liquid or solid dosage unit.

The doing regimen of this invention, as compared to previously taught dosing regimens comprising daily or twice daily administration of PPAR-alpha agonist, may result in no significant decrease in efficacy, for example control of lipids in the blood, but with a significant decrease in the risk for undesired effects, such liver, cardiac, or skeletal muscle toxicity. Or, for example, minimizing patient exposure to a potent PPAR-alpha agonist can further minimize the potential for any dosage related adverse reactions in the patient.

The dosing regimen of this invention may be used in conjunction with other treatments. For example, the PPAR-alpha agonist dosing regimen of this invention can be used in combination with a statin or drugs that increase insulin sensitization.

### DETAILED DESCRIPTION OF THE INVENTION

As used herein, a “PPAR-alpha agonist” includes any compound that selectively activates human PPAR-alpha as demonstrated by any accepted, validated assay for PPAR alpha activity, or any compound generally recognized as a PPAR-alpha agonist. See, for example, the PPAR-alpha assay methods described in the '553 patent. Such PPAR-alpha agonist may be an agonist of more than one PPAR subtype; however, the PPAR-alpha agonist will selectively activate PPAR-alpha to a significantly greater degree than PPAR gamma, PPAR delta, or PPAR gamma/delta dual activity. It is generally preferred that the PPAR alpha agonist is at least twice as potent agonist of PPAR alpha as for any other PPAR subtypes. Preferred PPAR-alpha agonists include, for example, those disclosed in the '553 patent. Particularly preferred are PPAR-alpha agonist compounds in clinical development, such as, the Eli Lilly and Company LY518674, Propanoic Acid, 2-[4-[3-[2,5-dihydro-1-[(4-methylphenyl)methyl]-5-oxo-1H-1,2,4-triazol-3-yl]propyl]phenoxy]-2-methyl-.

As used herein the term “SDSS” means single-blinded, single-placebo-controlled, dose-escalation study, and the term “MDSS” means multiple-dose placebo-controlled, dose-escalation study.

In addition to the typical daily drug dosing regimen, there are examples of drugs that use dosing regimens in which the interval between drug doses is more than a single day. While the interval may be two days (i.e. alternate-day dosing), or one week (i.e. once weekly dosing), or an extended period of no dosing following a period of daily dosing, the underlying principles are the same. In this discussion, any regimen intentionally comprising an inter-dose interval greater than one day is referred to as less than daily dosing.

As used herein, the compound LY518674 and Propanoic Acid, 2-[4-[3-[2,5-dihydro-1-[(4-methylphenyl)methyl]-5-oxo-1H-1,2,4-triazol-3-yl]propyl]phenoxy]-2-methyl-, each refer to a compound having the structural formula:.

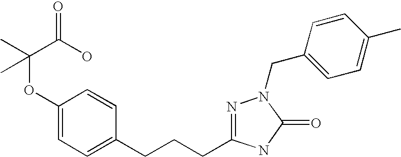

Less than daily dosing is known to be useful for certain pharmacological agents. For example, less than daily dosing is discussed in therapeutic areas in which glucocorticoid therapy is a mainstay. See, for example, Axelrod L., Glucocorticoids, Textbook of Rheumatology, 4^{th} edition, vol 1, pp 779-ff, (1986); Physicians Desk Reference, 48^{th} edition, section on DELTASONE, pp 2408-2409, (1994); Holland and Taylor, Glucocorticoids in clinical practice, J Family Prac 32: 512-519, (1991); and Hodson E M, Knight J R, Willis N S, and Craig J C, Corticosteroid therapy for nephrotic syndrome in children (Cochrane Review), The Cochrane Library, Issue 4, (2001).

Alendronate, used for prevention and treatment of osteoporosis, was originally developed for daily dosing. Subsequently, for various reasons, including minimization of esophagitis seen as a side effect, the drug was approved for once-a-week dosing. In this case, knowledge of pharmacokinetics specifically that the drug distributes only into bone was used to predict that infrequent dosing would be fully efficacious since bone is the target tissue for the drug. See, for example, Schnitzer, T J, Update on Alendronate: Once-Weekly dosing. Expert Opin Pharmacother 2: 1461-1472, (2001).

The clinical effect for the less than daily dosing regimen for PPAR alpha agonist appears to be independent of the drug pharmacokinetics. For example Propanoic Acid, 2-[4-[3-[2,5-dihydro-1-[(4-methylphenyl)methyl]-5-oxo-1H-1,2,4-triazol-3-yl]propyl]phenoxy]-2-methyl- is believed to be cleared from the body within hours; however, the beneficial effect of the PPAR alpha agonist continues even after the drug has effectively cleared from the body.

The PPAR alpha agonist, LY518674, is currently being studied for its effectiveness to slow progression of coronary artery atherosclerosis (reduction in atheroma volume percent change from baseline vs. placebo on a background of standard of care) using intravascular ultrasound, during a 2 year period. Additionally, LY518674 is being studied for its effectiveness to slow progression of atherosclerosis in the carotid artery using carotid intima-media thickness (CIMT), i.e, a statistically significant reduction in the progression of mean maximum CIMT vs. placebo on a background of standard of care during a 2 year period. The LY518674 compound is being studied for a statistically significant reduction in cardiovascular morbidity/mortality with a relative reduction in secondary prevention of major adverse cardiac events (MACE) vs. placebo on a background of standard of care during a 5 year period. LY518674 is also being studied for an association with a statistically significant decrease in biomarkers associated with vascular inflammation (e.g. hsCRP) verses placebo, and for regression of atherosclerosis as may be shown in vascular imaging trials. The LY518674 compound can be beneficial for slowing the progression of atherosclerosis through treatment of dyslipidemia in patients with low HDL and elevated triglycerides.

Pharmaceutical agents that are PPAR alpha agonists can be beneficial for improving insulin resistance (reducing homeostasis model assessment and qualitative insulin sensitivity check index). Additionally, PPAR alpha agonists can reduce serum free fatty acids, increase adiponectin, and decrease the local inflammatory response (high-sensitivity C-reactive protein [hsCRP], interleukin-6 [IL-6], and tumor necrosis factor alpha [TNF-]. Further, PPAR alpha agonists can lower levels of fasting triglycerides, and enhance atherosclerotic plaque stability that may further decrease the risk of ASCVD.

As used herein “Less than daily dosing” will be used to indicate any intentional dosing regimen that comprises at least one period in which the frequency of dosing is less than daily, for example every other day, every other week, or one time per month, or which comprises at least one gap of at more than 1 day where there is no administration of the PPAR-alpha agonist. In the case of dosing regimens that comprise a gap or day without administration, as used herein such gaps or days without administration of the PPAR-alpha agonist must be preceded and followed by administration of agonist once the initial dose has been administered. This is meant to include any dosing regimen comprising gaps in dosing of more than one day. For example, the dosing regimens of this invention include regimens comprising 1 day with and 2 days without, 1 day with and 5 days without, 1 day with and 7 days without, 1 day with and 6 days without, 1 day with and 14 days without, 1 day with and 13 days without, 1 day with and 21 days without, 1 day with and 20 days without, 1 day with and 30 days without, 1 day with and 28 days without, 1 day with and 11 days without, 1 day with and 22 days without, 1 day with and 1 day without, etc. This is true regardless of the average number of doses per day. For example, twice-daily administration every other day would be less than daily dosing. The dosing regimen of this invention can include several days or weeks of daily or twice daily dosing followed by a period of less than daily dosing. As used herein a day with dosing or a day with administration of agonist includes once, twice, or any number of doses on that particular day.

Preferred dosing regimens are those that comprise periods of once weekly dosing, or once every other week or that comprise six or thirteen consecutive days without administration. When the PPAR-alpha agonist dosing regimen of this invention is used in combination with administration of a statin or drug(s) that increase insulin sensitivity, or other additional drug(s) (collectively “combination treatment”), the combination treatment can be administered with a dosing regimen that is the same as or different from the PPAR-alpha agonist regimen. As used herein “dosing” and “administration” are intended to be identical.

As used herein the term “75 μg or less of Propanoic Acid, 2-[4-[3-[2,5-dihydro-1-[(4-methylphenyl)methyl]-5-oxo-1H-1,2,4-triazol-3-yl]propyl]phenoxy]-2-methyl-” means that the formulation contains no more than about 75 μg of Propanoic Acid, 2-[4-[3-[2,5-dihydro-1-[(4-methylphenyl)methyl]-5-oxo-1H-1,2,4-triazol-3-yl]propyl]phenoxy]-2-methyl- per dosage unit. It is generally preferred that each dosage unit will contain at least 1 μg Propanoic Acid, 2-[4-[3-[2,5-dihydro-1-[(4-methylphenyl)methyl]-5-oxo-1H-1,2,4-triazol-3-yl]propyl]phenoxy]-2-methyl- or a pharmaceutically acceptable salt thereof.

As used herein the term “LY518674” means Propanoic Acid, 2-[4-[3-[2,5-dihydro-1-[(4-methylphenyl)methyl]-5-oxo-1H-1,2,4-triazol-3-yl]propyl]phenoxy]-2-methyl- as the free acid or a pharmaceutical salt thereof. One preferred salt of Propanoic Acid, 2-[4-[3-[2,5-dihydro-1-[(4-methylphenyl)methyl]-5-oxo-1H-1,2,4-triazol-3-yl]propyl]phenoxy]-2-methyl- is the sodium salt.

### EXAMPLES

### Example 1

Low doses and/or a less than daily dosing regimen clinical study.

A single-blinded, single- (SDSS) and multiple-dose (MDSS), placebo-controlled, dose-escalation study is designed to evaluate the safety, pharmacokinetics, and pharmacodynamics of orally administered study drug in healthy obese men and women. Three different groups of subjects complete a one-step escalation of the study drug dose within Periods 1 and 2 (SDSS). The design includes a total of 6 active doses (0.1, 1, 10, 30, 60, and 100 mg) to be tested. The decision to escalate to higher doses is based on the safety assessment of preceding doses. The minimum time between dose escalations within Periods 1 and 2 is one week.

Each of the 3 groups of subjects consist of approximately 9 subjects in a 2:1 ratio of active treatment to placebo, where the order of placebo administration is randomized across both the SDSS and MDSS portions of the study. All 3 groups of subjects shall be scheduled to participate in both SDSS and MDSS portions. However, the SDSS portion is completed and analyzed several months prior to beginning the MDSS (Period 3/4). The 45-day MDSS portion of the original study is designed to test 4 active doses (0.025, 0.1, 1 and 10 mg) administered daily for the first 14 days of the study. The MDSS portion recruits subjects to form 4 groups of subjects. Approximately 36 subjects (9 per dose group) are enrolled into the MDSS portion of the study.

Safety is assessed throughout the study by physical examination, adverse event monitoring, electrocardiograms, clinical laboratory tests, and vital sign measurements. Pharmacodynamic evaluation includes a postprandial lipid response during the single-dose safety portion. During the multiple-dose safety study, the pharmacodynamic evaluation includes an oral glucose tolerance test, a fasting lipid panel and a postprandial lipid test. The static lipid panel included triglycerides, total cholesterol, low-density lipoprotein cholesterol [LDL-C], high-density lipoprotein cholesterol [HDL-C], Apoliproteins A1, A2, B, and C3 for cholesterol metabolism. C-reactive protein (CRP) and adiponectin are measured as biomarkers for inflammation and peroxisome proliferated agonist receptor (PPAR) gamma activity, respectively. The dynamic lipid tests include post-prandial measurements of glucose and triglycerides. The clinical study of Example 1 can be useful to demonstrate the effectiveness of low doses of study drug and/or less than daily dosing as claimed, respectively, herein.

### Results:

An SDSS clinical study was conducted substantially as described above by this Example 1 using LY518674 as the study drug. Gastrointestinal intolerance was assessed at 30 and 60 mg prohibiting administration of higher doses. Therefore, the 6^{th} dose (100 mg) was replaced by a 0.01 mg dose.

Patients receiving the 0.01 mg dose displayed an LDL-C reduction consistent with the reduction observed at the higher doses. ApoB and ApoC decreased in a fashion similar to that seen for LDL-C changes. The compound was rapidly absorbed after oral administration in healthy obese subjects. The median time at maximal concentration (tmax) was approximately 0.5 to 1 hour. The exposure appeared dose-proportional within the studied range. No differences were noted in the pharmacokinetics of the compound following single- and multiple-dose administration. Steady-state exposure of LY518674 was reached within 2 days, achieving minimal accumulation (mean accumulation ratio of no more than 1.35), consistent with a short half-life (less than 10 hours).

### Results of an SDSS Study:

A meal tolerance test (MTT) was performed from 24 to 37 hours after each single dose during the SDSS portion of a clinical trial conducted substantially as described herein by Example 1 using LY518674 as the study drug. The data are analyzed by calculating an area under the concentration-time curve for triglycerides, nonesterified fatty acids and glucose over the postprandial periods from breakfast to lunch (area under the effect curve [AUEC{24-28}]), after lunch (AUEC[28-37]), and the entire test period (AUEC[24-37]).

LY518674 had a rapid and profound reduction of triglyceride (TG) AUEC, as shown in Table 1 of this report. Furthermore, LY518674 exhibits a clear dose response that appears to approach a maximum AUEC reduction at approximately 1 mg. Remarkably, the effect continues throughout the test period as exhibited by the data obtained between 28 through 37 hours post dose.

TABLE 1
Comparison of Triglyceride AUEC (mmol · hr/L)
Between Study drug and Placebo from Meal Tolerance
Test in Single-Dose Portion

| SDSS Dynamic Lipid | | | Ratio of geometric means |
|---|---|---|---|
| TG | Dose | Geometric mean | Dose in row vs. Placebo |
| (mmol · hr/L) | (mg) | (95% CI) | (95% CI) [p-value] |
| AUEC(24-37) | Placebo | 44.6 (39.7, 50.1) | |
| | 0.01 | 37.8 (31.2, 45.8) | 0.85 (0.70, 1.02) [0.083] |
| | 0.1 | 27.6 (23.0, 33.2) | 0.62 (0.52, 0.74) [<.001*] |
| | 1 | 23.9 (20.0, 28.6) | 0.54 (0.45, 0.64) [<.001*] |
| | 10 | 24.3 (20.1, 29.5) | 0.55 (0.45, 0.67) [<.001*] |
| | 30 | 20.0 (16.3, 24.5) | 0.45 (0.37, 0.54) [<.001*] |
| | 60 | 25.2 (20.6, 30.7) | 0.56 (0.46, 0.69) [<.001*] |

Abbreviations: AUEC = area under the effect curve; CI = confidence interval; SDSS = single-dose safety study; TG = triglycerides.
*p-values < 0.05.

The results of this single dose study illustrate that LY518674 can provide a pharmaceutically desirable effect when dosed as low as 100 μg using a less than daily dosing regimen.

### Results of an MDSS Study

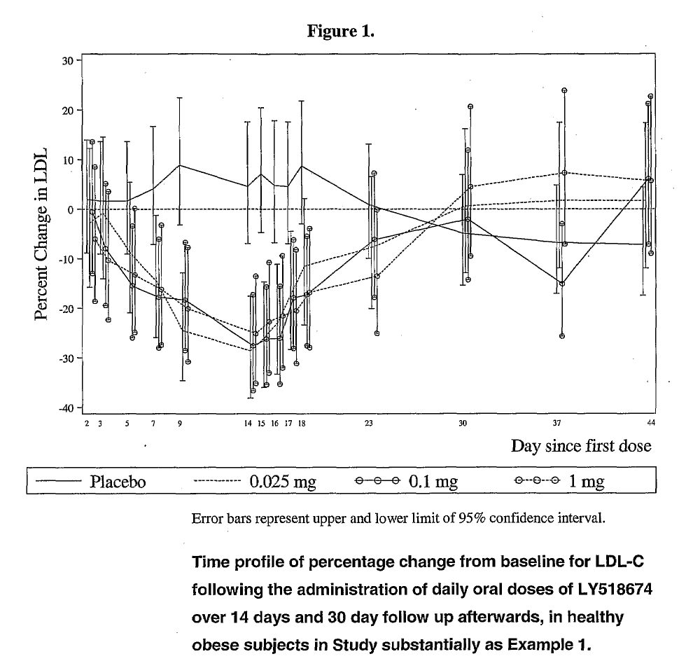

The data from treatment period (Days 1 through 14) and recovery period (Days 15 through 44) for LDL-C is depicted in FIG. 1. LDL-C was dramatically reduced by LY518674 with similar responses for total cholesterol and apolipoproteins B and C3. All doses produced similar magnitudes of change in these parameters. All parameters were back to baseline within 2 weeks after cessation of dosing. The maintenance of a pharmacological effect for nearly 14 days after dosing stopped, despite the half life of about 10 hours for LY518674 further supports that a less than daily dosing regimen may be desired for LY518674. The results of this study illustrate that LY518674 can provide a pharmaceutically desirable effect when dosed as low as 25 μg, using a less than daily dosing regimen.

### Example 2

The effectiveness of the low doses claimed herein can be further demonstrated by the following example.

This is a single- and multiple-dose, randomized, single-blind, placebo-controlled study to assess the safety, tolerability, pharmacokinetics and pharmacodynamics of orally Study drug in adult male subjects. This study consists of two portions for single dose and multiple doses. The doses selected for this study will be 1 μg, 10 μg, 25 μg and 100 μg. Subjects will be assigned to one of the 4 dose groups. Subjects will receive both single and multiple dose of study drug or placebo. Each subject will receive the same Study drug dose (or placebo) in their single and multiple dose periods. Each subject will have control day (Baseline day) with safety measures (ECGs, blood pressure and pulse rate) matched time on Day 1 or Day 19 before Study drug or placebo dose and will be admitted to the clinical research unit (CRU) on Day-1 (the day before dosing day of single dose period) and remain in the CRU until Day 22. Subjects will be discharged on Day 22 after safety evaluation. After discharged, subjects will visit the CRU for the follow-up evaluation between 5 and 10 days after last dose.

On Day 1, subjects will receive single doses of study drug (or placebo) and On Day 6, subjects will start the 1^{st} dosing for multiple dose period. Subjects receive the study drug (or placebo) every morning on Day 7-19. (Table 1).

TABLE 1
Dosing Schedule

| | Number of | Single dose period | Multiple Dose Period |
|---|---|---|---|
| Group^{a} | Subject | Day 1 | Day 6-19 |
| 1 | 8 | Study drug 1 μg | Study drug 1 μg |
| | 2 | Placebo | Placebo |
| 2 | 8 | Study drug 10 μg | Study drug 10 μg |
| | 2 | Placebo | Placebo |
| 3 | 8 | Study drug 25 μg | Study drug 25 μg |
| | 2 | Placebo | Placebo |
| 4 | 8 | Study drug 100 μg | Study drug 100 μg |
| | 2 | Placebo | Placebo |

^{a}It is intended that 40 subjects will be enrolled.

It is intended that up to 52 subjects may be enrolled into 4 dose groups to complete a minimum of 32 subjects (8 per group). The multiple doses of study drug should be started after obtaining safety information from Day 1 to the morning of Day 6 in all subjects (8 active and 2 placebo) at each group. The single dose administration for the next dose group should be given after obtaining safety information at final dosing (Day 19) from all subjects for previous group. Safety will be assessed throughout the study. Blood samples will be collected from all subjects receiving active study drug and placebo.

The Example 2 clinical study can be conducted substantially as described herein above in Japanese male subjects using LY518674 as the study drug. This study design can be used to demonstrate the clinical effectiveness of a dosage containing 25 μg, 10 μg and 1 μg respectively of LY518674.

### Example 3

Less than Daily Dosing Pre-Clinical Study

Studies will be performed in human apoA-I transgenic mice to characterize the ability of Study drug to increase HDL-C and to lower serum triglycerides in a less than daily dosing paradigm. Animals will be divided into groups of six and dosed with 0.3 mg/kg Study drug either daily, every-other day, every three days or once a week for two weeks by oral gavage with suspensions of the free acid or with vehicle (1% wt/v CMC, 0.25% Tween 80). Animals will be bled by cardiac puncture following euthanasia with CO₂. Serum will be prepared for determination of HDL-cholesterol by fast protein liquid chromatography (FPLC) and for determination of total serum cholesterol and triglycerides by enzymatic analysis from individual animals. Triglyceride and HDL-cholesterol levels from animals administered less than daily dosing Study drug will be compared to levels obtained from animals administered the daily dosing paradigm.

Effects of a less than daily dosing paradigm of Study drug on LDL-cholesterol will be determined using Golden Syrian hamsters fed a high-fat/high-cholesterol diet (10% coconut oil, 0.12% cholesterol). Animals will be divided into groups of six and dosed with 3 mg/kg Study drug either daily, every-other day, every three days or once a week for two weeks by gavage with suspensions of the free acid or with vehicle (1% wt/v CMC, 0.25% Tween 80). Animals will be bled by cardiac puncture following euthanasia with CO₂. Serum will be prepared for determination of LDL-cholesterol by fast protein liquid chromatography (FPLC). LDL-cholesterol levels from animals administered less than daily dosing Study drug will be compared to levels obtained from animals administered the daily dosing paradigm.

The results of these studies can be useful to demonstrate that triglyceride-lowering, HDL-raising, and LDL-lowering efficacy mediated by LY518674 may be achieved with less than daily dose administration.

### Example 4

Clinical Simulation of Less than Daily Dosing Using Results from a Clinical Trial-MDSS Study Completed Substantially as Described in Example 1.

The effectiveness of less than daily dosing claimed herein can be further demonstrated through mathematical simulations. A model was built based on MDSS data that related the time course of drug concentration to the time course of LDL reduction. The relationship is based on a hypothesized stimulation of LDL clearance as the mechanism of action for LY518674 LDL reducing effect. The model enabled simulations of many different scenarios of dosing regimens. To illustrate the effectiveness of less than daily doses, the following 90-day dosing scenarios were simulated and compared to placebo: Once daily dosing, 6 once-daily dosing periods interrupted by one dose-free day, 2 once-daily dosing periods interrupted by one dose-free day, once every-other-day dosing, 1 dose every 3 days, 1 dose every week. The total dose administration was kept the same across the dosing scenarios, where the daily dosing amount during the dosing periods for the less-than-daily dosing scenarios was increased compared to the once-daily dosing scenario. The starting baseline LDL-C was 142 mg/dL, which was observed in the MDSS study. The dose used in the mathematical simulations was 100 μG for the once-daily dosing scenario. The methods used to create the mathematical simulations reported herein are consistent with typical methods known to the skilled artisan.

### Results.

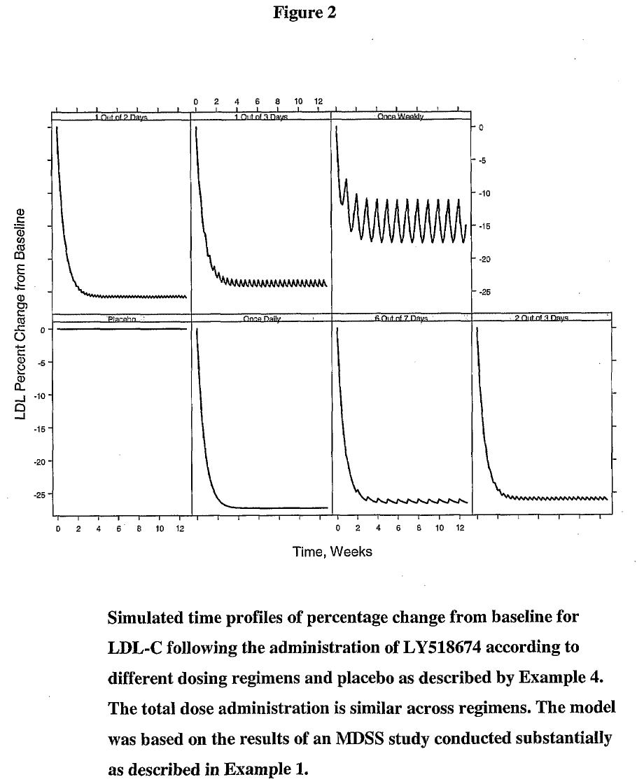

The model-predicted time-course of LDL is depicted in FIG. 2. The area under LDL time course (AUEC-LDL) represents the total effectiveness, and when compared after 3 weeks from the start of therapy it represents steady-state behavior. The AUEC-LDL from 3 to 13 weeks after the start of therapy was calculated for the aforementioned scenarios and is presented in Table 3. As seen from FIG. 2 and Table 3, all dosing regimens provide clear effectiveness, to varying degrees, compared to placebo.

TABLE 3
The area under the LDL-C time curve from 3 weeks to
13 weeks of administration in comparison to placebo.

| | % Change in AUEC-LDL from |
|---|---|
| Scenario | placebo |
| Placebo | 0 |
| Once Daily | −27 |
| 6 Out of 7 Days | −26 |
| 2 Out of 3 Days | −26 |
| 1 Out of 2 Days | −26 |
| 1 Out of 3 Days | −24 |
| Once Weekly | −15 |

What is claimed is:

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A kit comprising at least one pharmaceutically effective dosage unit of Propanoic Acid, 2-[4-[3-[2,5-dihydro-1-[(4-methylphenyl)methyl]-5-oxo-1H-1,2,4-triazol-3-yl]propyl]phenoxy]-2-methyl- or a salt thereof, for administration according to a continuous schedule having a dosing interval selected from the group consisting of once-weekly dosing and bi-weekly dosing.

19. A dosage unit comprising about 75 μg or less Propanoic Acid, 2-[4-[3-[2,5-dihydro-1-[(4-methylphenyl)methyl]-5-oxo-1H-1,2,4-triazol-3-yl]propyl]phenoxy]-2-methyl- or a salt thereof, and at least one pharmaceutically acceptable carrier or excipient.

20. A dosage unit as claimed by claim 19 wherein the dosage unit is a formulation comprising less than about 50 μg Propanoic Acid, 2-[4-[3-[2,5-dihydro-1-[(4-methylphenyl)methyl]-5-oxo-1H-1,2,4-triazol-3-yl]propyl]phenoxy]-2-methyl- or a salt thereof.

21. A dosage unit as claimed by claim 20 wherein the dosage unit is a formulation comprising less than about 25 μg Propanoic Acid, 2-[4-[3-[2,5-dihydro-1-[(4-methylphenyl)methyl]-5-oxo-1H-1,2,4-triazol-3-yl]propyl]phenoxy]-2-methyl- or a salt thereof.

22. A dosage unit as claimed by claim 21 wherein the dosage unit is a formulation comprising about 25 μg Propanoic Acid, 2-[4-[3-[2,5-dihydro-1-[(4-methylphenyl)methyl]-5-oxo-1H-1,2,4-triazol-3-yl]propyl]phenoxy]-2-methyl- or a salt thereof.

23. A dosage unit as claimed claim 21 wherein the dosage unit is a solid formulation.

24. A dosage unit as claimed by claim 21 wherein the dosage unit is a formulation comprising from about 10 μg to about 25 μg Propanoic Acid, 2-[4-[3-[2,5-dihydro-1-[(4-methylphenyl)methyl]-5-oxo-1H-1,2,4-triazol-3-yl]propyl]phenoxy]-2-methyl- or a salt thereof.

25. A dosage unit as claimed by claim 21 wherein the dosage unit is a formulation comprising from about 5 μg to about 10 μg Propanoic Acid, 2-[4-[3-[2,5-dihydro-1-[(4-methylphenyl)methyl]-5-oxo-1H-1,2,4-triazol-3-yl]propyl]phenoxy]-2-methyl- or a salt thereof.

26. A dosage unit as claimed by claim 21 wherein the dosage unit is a formulation comprising less than about 5 μg Propanoic Acid, 2-[4-[3-[2,5-dihydro-1-[(4-methylphenyl)methyl]-5-oxo-1H-1,2,4-triazol-3-yl]propyl]phenoxy]-2-methyl- or a salt thereof.

27. A dosage unit as claimed by claim 21 wherein the dosage unit is a formulation comprising about 1 μg Propanoic Acid, 2-[4-[3-[2,5-dihydro-1-[(4-methylphenyl)methyl]-5-oxo-1H-1,2,4-triazol-3-yl]propyl]phenoxy]-2-methyl- or a salt thereof.

28. A dosage unit as claimed by claim 2 wherein the dosage unit is a pharmaceutical formulation.

29. A dosage unit as claimed by claim 21 wherein the dosage unit is a tablet or capsule formulation.

30. A dosage unit as claimed by claim 29 wherein the dosage unit is a tablet formulation.

31. A dosage unit as claimed by claim 21 wherein the dosage unit is an oral formulation.

32. A dosage unit as claimed by claim 21 wherein the active Propanoic Acid, 2-[4-[3-[2,5-dihydro-1-[(4-methylphenyl)methyl]-5-oxo-1H-1,2,4-triazol-3-yl]propyl]phenoxy]-2-methyl- is the free acid.

33. A dosage unit as claimed by claim 21 wherein the dosage unit is a formulation comprising from about 5 μg to about 25 μg Propanoic Acid, 2-[4-[3-[2,5-dihydro-1-[(4-methylphenyl)methyl]-5-oxo-1H-1,2,4-triazol-3-yl]propyl]phenoxy]-2-methyl- or a salt thereof
